(19) United States
(12) Patent Application Publication
(10) Pub. No.: US 2024/0058488 A1
(43) Pub. Date: Feb. 22, 2024

(54) METHOD AND APPARATUS FOR DISINFECTING LABORATORY INSTRUMENTS

(71) Applicants: Thomas Forest FARB, Wakefield, MA (US); Leslie Alana STONE MEISEL, Santa Monica, CA (US); THRIVE BIOSCIENCE, INC.,, Wakefield, MA (US)

(72) Inventors: Thomas Forest FARB, Wakefield, MA (US); Leslie Alana STONE MEISEL, Santa Monica, CA (US)

(73) Assignee: THRIVE BIOSCIENCE, INC., Wakefield, MA (US)

(21) Appl. No.: 18/259,777

(22) PCT Filed: Dec. 30, 2021
(86) PCT No.: PCT/US2021/065606

§ 371 Date: Jun. 29, 2023

Related U.S. Application Data
(60) Provisional application No. 63/132,678, filed on Dec. 31, 2020.

(51) Int. Cl. A61L 2/10; A61L 2/26

(52) U.S. Cl. CPC A61L 2/10; A61L 2/26; A61L 2202/14

(57) ABSTRACT

A method and apparatus for disinfecting a laboratory instrument having a housing with pathogens therein. A plurality of UVC light sources are provided in the housing and the pathogens in the housing are moved relative to the light from the UVC light sources.

### PRIORITY CLAIM

This application claims priority of U.S. Provisional Application Ser. No. 63/132,678, filed Dec. 31, 2020, the entire contents of which are hereby incorporated herein by reference.

### FIELD OF THE INVENTION

The present invention relates to laboratory instruments and in particular to imaging systems such as imaging systems for cell cultures.

### BACKGROUND

Cell culture incubators are used to grow and maintain cells from cell culture, which is the process by which cells are grown under controlled conditions. Cell culture vessels containing cells are stored within the incubator, which maintains conditions such as temperature and gas mixture that are suitable for cell growth. Cell imagers take images of individual or groups of cells for cell analysis.

Long-term cell culture is a useful technique in both research and clinical contexts. However, maintenance of long-term cell cultures, for example, long term cultures, tissue preparations, in vitro fertilization preparations, etc., in presently available cell incubators is a laborious process requiring highly trained personnel and stringent aseptic conditions.

While scientists use microscopes to observe cells during culturing and may also attach a camera to the microscope to image cells in a cell culture, such imaging systems have many disadvantages.

Cell imagers are usually placed in laboratories where pathogens might be inadvertently released into the air or onto surfaces, or there may be an inadvertent spill of pathogens into the imager itself during the insertion or removal of a cell culture vessel. In those instances, it is desirable to disinfect the instrument before doing any further imaging so as not to contaminate other cell cultures and to protect users of the imager.

### SUMMARY

The object of the present invention is to improve laboratory instruments and in particular imaging systems for imaging cells in a cell culture. An imaging system and method of this type is described in U.S. application Ser. No. 15/563,375 filed on Mar. 31, 2016 and the disclosure of which in its entirety is hereby incorporated by reference.

Another object is to provide a method and an apparatus for disinfecting the imaging system. Ultraviolet light (UV) that operates at wavelengths between 200-280 nanometers (nm) is typically described as being in the UV “C” band. The light in this type of spectrum is not visible to the human eye and can be used to kill or inactivate pathogens in an imaging system. UVC lights that can be used in accordance with the inventions described herein are identified in IES CR-2-20-V1 “IES Committee Report: Germicidal Ultraviolet GUV)—Frequently Asked Questions,” Illuminating Engineering Society 2020, the disclosure of which in its entirety is hereby incorporated by reference.

Because not all UVC lamps are the same, lamps may emit very specific UVC wavelengths (e.g., 254 nm or 222 nm), or they may emit a broad range of UV wavelengths (e.g, 200 nm to 280 nm). Some lamps also emit visible and infrared radiation. The wavelengths emitted by the lamp may affect the lamp's effectiveness at inactivating a particular virus or killing another specific pathogen, and therefore a variety of lamps may be used in certain laboratory conditions where a variety of pathogens my appear. Some lamps emit multiple types of wavelengths and may therefore be used instead of multiple lamps. The UVC wavelength range generally considered to encompass the peak UVC germicidal wavelength is 265 nm to 270 nm. The UVC wavelengths generally considered best to deactivate SARS-CoV-2 are 254 nm and 222 nm. Although UVC at 254 nm is generally considered more efficient than UVC at 222 nm for deactivating SARS-CoV-2, UVC at 222 nm is generally considered to be less harmful to human tissue than is UVC at 254 nm.

Lamps that may be used include low-pressure mercury lamps, excimer lamps, pulsed xenon lamps, and light emitting diodes (LEDs). The LEDs are particularly useful in an imager because they are low voltage compact devices that can be strategically mounted within the imager to provide coverage of most of the internal surfaces of the imager.

In some embodiments, the effectiveness of the lamps can be improved by adding air movement devices such as fans or blowers to move the air and therefore the pathogens into the path of the UVC light. Alternatively, moving mirrors and stationary mirrors and other stationary reflective surfaces can be used to reflect the light over more surfaces and spaces. In other embodiments, any or all of mirrors, other reflective surfaces, and air movers can be used together and can be combined, for example, a fan with reflective blades.

In other embodiments, the use of UVC light in combination with one or more gases or liquids can be used to disinfect.

In some embodiments, the imaging system and method described herein can be used as a stand-alone imaging system or it can be integrated in a cell incubator using a transport described in the aforementioned application incorporated by reference. In some embodiments, the imaging system and method is integrated in a cell incubator and includes a transport.

In some embodiments the system and method acquire data and images at the times a cell culturist typically examines cells. The method and system provide objective data, images, guidance and documentation that improves cell culture process monitoring and decision-making.

The system and method in some embodiments enable sharing of best practices across labs, assured repeatability of process across operators and sites, traceability of process and quality control. In some embodiments the method and system provide quantitative measures of cell doubling rates, documentation and recording of cell morphology, distribution and heterogeneity.

In some embodiments, the method and system provide assurance that cell lines are treated consistently, and that conditions and outcomes are tracked. In some embodiments the method and system learn through observation, and record how different cells grow under controlled conditions in an onboard database. Leveraging this database of observations, researchers are able to profile cell growth, test predictions and hypotheses concerning cell conditions, media and other factors affecting cell metabolism, and determine whether cells are behaving consistently and/or changing.

In some embodiments the method and system enable routine and accurate confluence measurements and imaging and enables biologists to quantify responses to stimulus or intervention, such as the administration of a therapeutic to a cell line.

The method and system capture the entire well area with higher coverage than conventional images and enables the highest level of statistical rigor for quantifying cell status and distribution.

In some embodiments, the method and system provide image processing and algorithms that will deliver an integration of individual and group morphologies with process-flow information and biological outcomes. Full well imaging allows the analysis and modeling of features of groups of cells—conducive to modeling organizational structures in biological development. These capabilities can be used for prediction of the organizational tendency of a culture in advance of functional testing.

In some embodiments, algorithms are used to separate organizational patterns between samples using frequency of local slope field inversions. Using some algorithms, the method and system can statistically distinguish key observed differences between iP-MSCs generated from different TCP conditions. Biologically, this work could validate serum-free differentiation methods for iPSC MSC differentiation. Computationally, the method and system can inform image-processing of MSCs in ways that less neatly “clustered” image sets are not as qualified to do.

Even if all iP-MSC conditions have a sub-population of cells that meets ISCT 7-marker criteria, the “true MSC” sub-populations may occupy a different proportion under different conditions or fate differences could be implied by tissue “meso-structures”. By starting with a rich pallet of MSC outcomes, and grounding them in comparative biological truth, the method and system can refine characterization perspectives around this complex cell type and improve MSC bioprocess.

In certain embodiments, an imager includes one or more lenses, fibers, cameras (e.g., a charge-coupled device camera), apertures, mirrors, light sources (e.g., a laser or lamp), or other optical elements. An imager may be a microscope. In some embodiments, the imager is a bright-field microscope. In other embodiments, the imager is a holographic imager or microscope. In other embodiments the imager is a phase-contrast microscope. In other embodiments, the imager is a fluorescence imager or microscope.

As used herein, the “fluorescence imager” is an imager which is able to detect light emitted from fluorescent markers present either within or on the surface of cells or other biological entities, said markers emitting light in a specific wavelength when absorbing a light of different specific excitation wavelength.

As used herein, a “bright-field microscope” is an imager that illuminates a sample and produces an image based on the light absorbed by the sample. Any appropriate bright-field microscope may be used in combination with an incubator provided herein.

As used herein, a “phase-contrast microscope” is an imager that converts phase shifts in light passing through a transparent specimen to brightness changes in the image. Phase shifts themselves are invisible but become visible when shown as brightness variations. Any appropriate phase-contrast microscope may be used in combination with an incubator provided herein.

As used herein, a “holographic imager” is an imager that provides information about an object (e.g., sample) by measuring both intensity and phase information of electromagnetic radiation (e.g., a wave front). For example, a holographic microscope measures both the light transmitted after passing through a sample as well as the interference pattern (e.g., phase information) obtained by combining the beam of light transmitted through the sample with a reference beam.

A holographic imager may also be a device that records, via one or more radiation detectors, the pattern of electromagnetic radiation, from a substantially coherent source, diffracted or scattered directly by the objects to be imaged, without interfering with a separate reference beam and with or without any refractive or reflective optical elements between the substantially coherent source and the radiation detector(s).

### Holographic Microscopy

In some embodiments, holographic microscopy is used to obtain images (e.g., a collection of three-dimensional microscopic images) of cells for analysis (e.g., cell counting) during culture (e.g., long-term culture) in an incubator (e.g., within an internal chamber of an incubator as described herein). In some embodiments, a holographic image is created by using a light field, from a light source scattered off objects, which is recorded and reconstructed. In some embodiments, the reconstructed image can be analyzed for a myriad of features relating to the objects. In some embodiments, methods provided herein involve holographic interferometric metrology techniques that allow for non-invasive, marker-free, quick, full-field analysis of cells, generating a high resolution, multi-focus, three-dimensional representation of living cells in real time.

In some embodiments, holography involves shining a coherent light beam through a beam splitter, which divides the light into two equal beams: a reference beam and an illumination beam. In some embodiments, the reference beam, often with the use of a mirror, is redirected to shine directly into the recording device without contacting the object to be viewed. In some embodiments, the illumination beam is also directed, using mirrors, so that it illuminates the object, causing the light to scatter. In some embodiments, some of the scattered light is then reflected onto the recording device. In some embodiments, a laser is generally used as the light source because it has a fixed wavelength and can be precisely controlled. In some embodiments, to obtain clear images, holographic microscopy is often conducted in the dark or in low light of a different wavelength than that of the laser in order to prevent any interference. In some embodiments, the two beams reach the recording device, where they intersect and interfere with one another. In some embodiments, the interference pattern is recorded and is later used to reconstruct the original image. In some embodiments, the resulting image can be examined from a range of different angles, as if it was still present, allowing for greater analysis and information attainment.

In some embodiments, digital holographic microscopy is used in incubators described herein. In some embodiments, digital holographic microscopy light wave front information from an object is digitally recorded as a hologram, which is then analyzed by a computer with a numerical reconstruction algorithm. In some embodiments, the computer algorithm replaces an image forming lens of traditional microscopy. The object wave front is created by the object's illumination by the object beam. In some embodiments, a microscope objective collects the object wave front, where the two wave fronts interfere with one another, creating the hologram. Then, the digitally recorded hologram is transferred via an interface (e.g., IEEE1394, Ethernet, serial) to a PC-based numerical reconstruction algorithm, which results in a viewable image of the object in any plane.

In some embodiments, in order to procure digital holographic microscopic images, specific materials are utilized. In some embodiments, an illumination source, generally a laser, is used as described herein. In some embodiments, a Michelson interferometer is used for reflective objects. In some embodiments, a Mach-Zehnder interferometer for transmissive objects is used. In some embodiments, interferometers can include different apertures, attenuators, and polarization optics in order to control the reference and object intensity ratio. In some embodiments, an image is then captured by a digital camera, which digitizes the holographic interference pattern. In some embodiments, pixel size is an important parameter to manage because pixel size influences image resolution. In some embodiments, an interference pattern is digitized by a camera and then sent to a computer as a two-dimensional array of integers with 8-bit or higher grayscale resolution. In some embodiments, a computer's reconstruction algorithm then computes the holographic images, in addition to pre- and post-processing of the images.

### Phase Shift Image

In some embodiments, in addition to the bright field image generated, a phase shift image results. Phase shift images, which are topographical images of an object, include information about optical distances. In some embodiments, the phase shift image provides information about transparent objects, such as living biological cells, without distorting the bright field image. In some embodiments, digital holographic microscopy allows for both bright field and phase contrast images to be generated without distortion. Also, both visualization and quantification of transparent objects without labeling is possible with digital holographic microscopy. In some embodiments, the phase shift images from digital holographic microscopy can be segmented and analyzed by image analysis software using mathematical morphology, whereas traditional phase contrast or bright field images of living unstained biological cells often cannot be effectively analyzed by image analysis software.

In some embodiments, a hologram includes all of the information pertinent to calculating a complete image stack. In some embodiments, since the object wave front is recorded from a variety of angles, the optical characteristics of the object can be characterized, and tomography images of the object can be rendered. From the complete image stack, a passive autofocus method can be used to select the focal plane, allowing for the rapid scanning and imaging of surfaces without any vertical mechanical movement. Furthermore, a completely focused image of the object can be created by stitching the sub-images together from different focal planes. In some embodiments, a digital reconstruction algorithm corrects any optical aberrations that may appear in traditional microscopy due to image-forming lenses. In some embodiments, digital holographic microscopy advantageously does not require a complex set of lenses; but rather, only inexpensive optics, and semiconductor components are used in order to obtain a well-focused image, making it relatively lower cost than traditional microscopy tools.

### Applications

In some embodiments, holographic microscopy can be used to analyze multiple parameters simultaneously in cells, particularly living cells. In some embodiments, holographic microscopy can be used to analyze living cells, (e.g., responses to stimulated morphological changes associated with drug, electrical, or thermal stimulation), to sort cells, and to monitor cell health. In some embodiments, digital holographic microscopy counts cells and measures cell viability directly from cell culture plates without cell labeling. In other embodiments, the imager can be used to examine apoptosis in different cell types, as the refractive index changes associated with the apoptotic process can be quantified via digital holographic microscopy. In some embodiments, digital holographic microscopy is used in research regarding the cell cycle and phase changes. In some embodiments, dry cell mass (which can correlate with the phase shift induced by cells), in addition to other non-limiting measured parameters (e.g., cell volume, and the refractive index), can be used to provide more information about the cell cycle at key points.

In some embodiments, the method is also used to examine the morphology of different cells without labeling or staining. In some embodiments, digital holographic microscopy can be used to examine the cell differentiation process; providing information to distinguish between various types of stem cells due to their differing morphological characteristics. In some embodiments, because digital holographic microscopy does not require labeling, different processes in real time can be examined (e.g., changes in nerve cells due to cellular imbalances).

In some embodiments, cell volume and concentration may be quantified, for example, through the use of digital holographic microscopy's absorption and phase shift images. In some embodiments, phase shift images may be used to provide an unstained cell count. In some embodiments, cells in suspension may be counted, monitored, and analyzed using holographic microscopy.

In some embodiments, the time interval between image acquisitions is influenced by the performance of the image recording sensor. In some embodiments, digital holographic microscopy is used in time-lapse analyses of living cells. For example, the analysis of shape variations between cells in suspension can be monitored using digital holographic images to compensate for defocus effects resulting from movement in suspension. In some embodiments, obtaining images directly before and after contact with a surface allows for a clear visual of cell shape. In some embodiments, a cell's thickness before and after an event can be determined through several calculations involving the phase contrast images and the cell's integral refractive index. Phase contrast relies on different parts of the image having different refractive index, causing the light to traverse different areas of the sample with different delays. In some embodiments, such as phase contrast microscopy, the out of phase component of the light effectively darkens and brightens particular areas and increases the contrast of the cell with respect to the background. In some embodiments, cell division and migration are examined through time-lapse images from digital holographic microscopy. In some embodiments, cell death or apoptosis may be examined through still or time-lapse images from digital holographic microscopy.

In some embodiments, digital holographic microscopy can be used for tomography, including but not limited to, the study of subcellular motion, including in living tissues, without labeling.

In some embodiments, digital holographic microscopy does not involve labeling and allows researchers to attain rapid phase shift images, allowing researchers to study the minute and transient properties of cells, especially with respect to cell cycle changes and the effects of pharmacological agents.

These and other features and advantages, which characterize the present non-limiting embodiments, will be apparent from a reading of the following detailed description and a review of the associated drawings. It is to be understood that both the foregoing general description and the following detailed description are explanatory only and are not restrictive of the non-limiting embodiments as claimed.

### BRIEF DESCRIPTION OF THE DRAWINGS

FIG. 1 is a perspective view of the imaging system according to the invention;

FIG. 2 is the imaging system of FIG. 1 with walls removed to reveal the internal structure;

FIG. 3 is a top view of the imaging system of FIG. 1 with the walls removed;

FIG. 4 is a right side view of the imaging system of FIG. 1;

FIG. 5 is a left side view of the imaging system of FIG. 1;

FIG. 6 is a block diagram of the circuitry of the imaging system of FIG. 1; and

FIG. 7 shows the left side view modified with the placement of UVC LEDs, fans, blowers, mirrors and other reflective surfaces.

### DETAILED DESCRIPTION

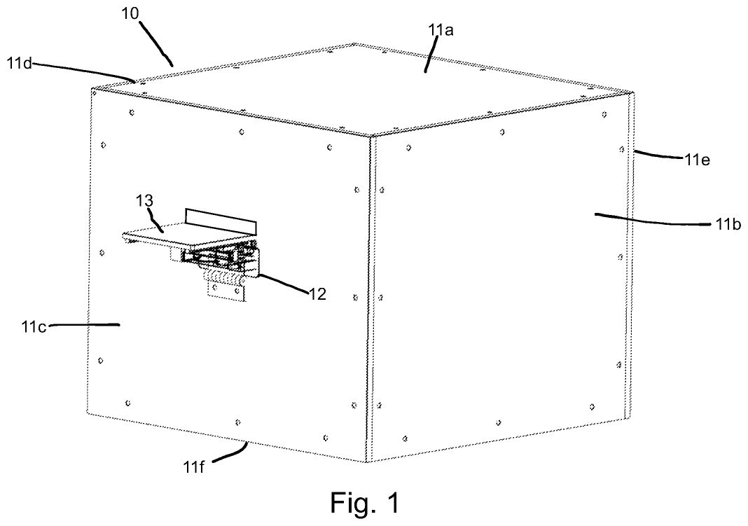

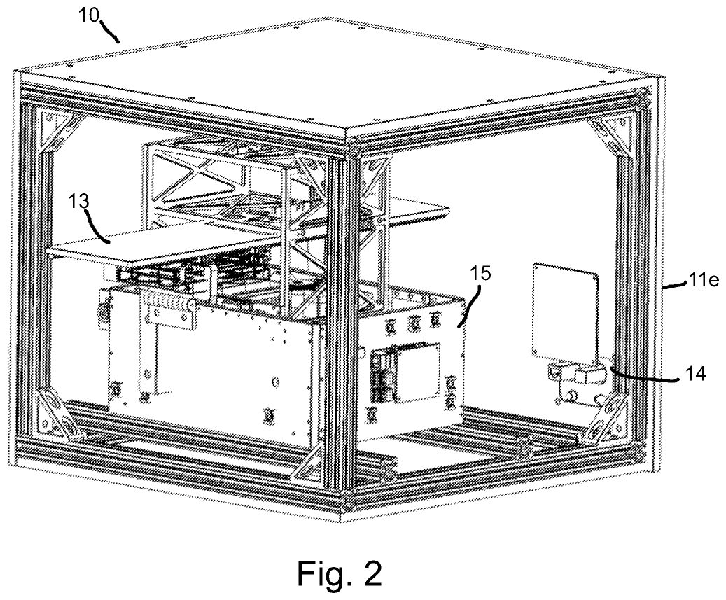

Referring now to FIG. 1, a cell imaging system 10 is shown. Preferably, the system 10 is fully encased with walls 11a-11f so that the interior of the imager can be set at 98.6 degrees F. with a CO₂ content of 5%, so that the cells can remain in the imager without damage. The temperature and the CO₂ content of the air in the system 10 is maintained by a gas feed port 14 (shown in FIG. 2) in the rear wall 11e. Alternatively, a heating unit can be installed in the system 10 to maintain the proper temperature.

At the front wall 11c of the system 10, is a door 12 that is hinged to the wall 11c and which opens when sliding platform 13 exits to receive a plate and closes when the platform 13 is retracted into the system 10.

The system 10 can also be connected to a computer or tablet for data input and output and for the control of the system. The connection is by way of an ethernet connector 15 in the rear wall 11e of the system as shown in FIG. 2.

FIG. 2 shows the system with walls 11b and 11c removed to show the internal structure. The extent of the platform 13 is shown as well as the circuit board 15 that contains much of the circuitry for the system, as will be explained in more detail hereinafter.

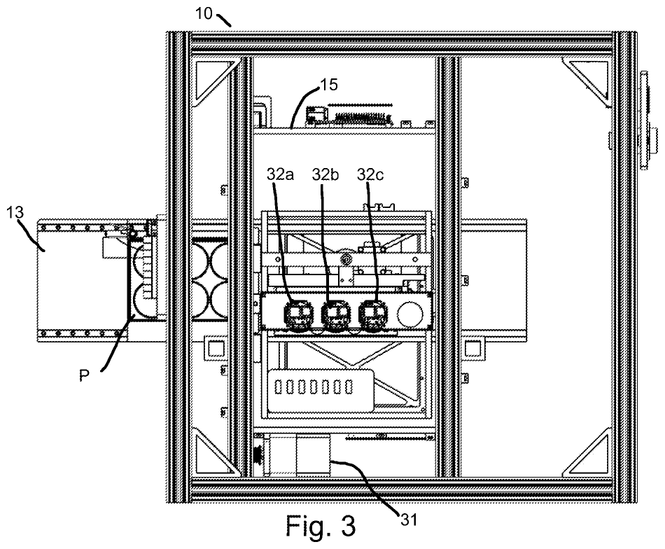

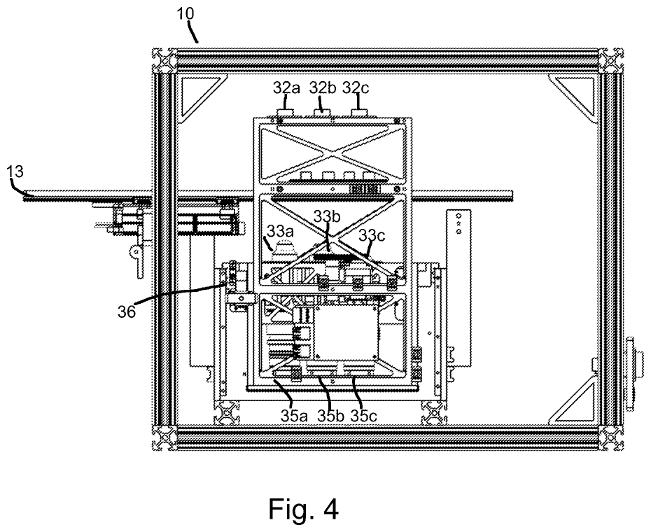

FIG. 3 shows a top view of the imaging system where plate P having six wells is loaded for insertion into the system on platform 13. Motor 31 draws the platform 13 and the loaded plate P into the system 10. The motor 31 moves the platform 13 in both the X-direction into and out of the system and in the Y-direction by means of a mechanical transmission 36. The movement of the platform is to cause each of the wells to be placed under one of the LED light clusters 32a, 32b, and 32c which are aligned with microscope optics 33a, 33b and 33c respectively which are preferably 4×, 10× and 20× phase-contrast and brightfield optics which are shown in FIG. 4.

As used herein, an “imager” refers to an imaging device for measuring light (e.g., transmitted or scattered light), color, morphology, or other detectable parameters such as a number of elements or a combination thereof. An imager may also be referred to as an imaging device. In certain embodiments, an imager includes one or more lenses, fibers, cameras (e.g., a charge-coupled device or CMOS camera), apertures, mirrors, light sources (e.g., a laser or lamp), or other optical elements. An imager may be a microscope. In some embodiments, the imager is a bright-field microscope. In other embodiments, the imager is a holographic imager or microscope. In other embodiments, the imager is a fluorescence microscope.

As used herein, a “fluorescence microscope” refers to an imaging device which is able to detect light emitted from fluorescent markers present either within and/or on the surface of cells or other biological entities, said markers emitting light at a specific wavelength in response to the absorption a light of a different wavelength.

As used herein, a “bright-field microscope” is an imager that illuminates a sample and produces an image based on the light absorbed by the sample. Any appropriate bright-field microscope may be used in combination with an incubator provided herein.

As used herein, a “holographic imager” is an imager that provides information about an object (e.g., sample) by measuring both intensity and phase information of electromagnetic radiation (e.g., a wave front). For example, a holographic microscope measures both the light transmitted after passing through a sample as well as the interference pattern (e.g., phase information) obtained by combining the beam of light transmitted through the sample with a reference beam.

A holographic imager may also be a device that records, via one or more radiation detectors, the pattern of electromagnetic radiation, from a substantially coherent source, diffracted or scattered directly by the objects to be imaged, without interfering with a separate reference beam and with or without any refractive or reflective optical elements between the substantially coherent source and the radiation detector(s).

In some embodiments, an incubator cabinet includes a single imager. In some embodiments, an incubator cabinet includes two imagers. In some embodiments, the two imagers are the same type of imager (e.g., two holographic imagers or two bright-field microscopes). In some embodiments, the first imager is a bright-field microscope and the second imager is a holographic imager. In some embodiments, an incubator cabinet comprises more than 2 imagers. In some embodiments, cell culture incubators comprise three imagers. In some embodiments, cell culture incubators having 3 imagers comprise a holographic microscope, a bright-field microscope, and a fluorescence microscope.

As used herein, an “imaging location” is the location where an imager images one or more cells. For example, an imaging location may be disposed above a light source and/or in vertical alignment with one or more optical elements (e.g., lens, apertures, mirrors, objectives, and light collectors).

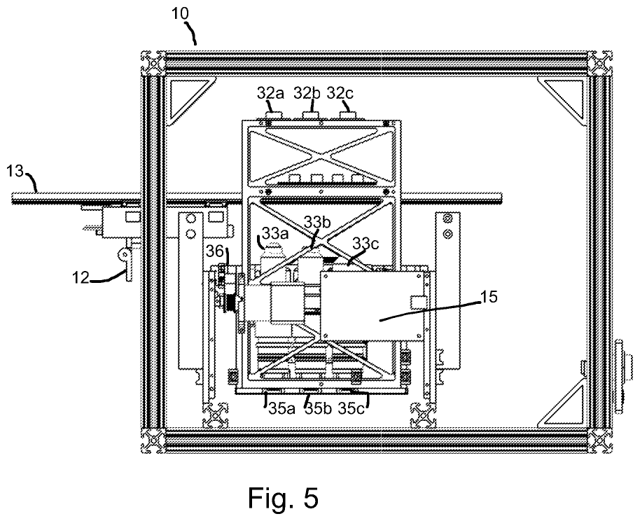

Referring to FIGS. 4-5, Under the control of the circuitry on board 15, each well is aligned with a desired one of the three optical units 33a-33c and the corresponding LED is turned on for brightfield illumination. The image seen by the optical unit is recorded by the respective video camera 35a, 35b, and 35c corresponding to the optical unit. The imaging and the storing of the images are all under the control of the circuitry on board 15. After the imaging is completed, the platform with the loaded plate is ejected from the system and the plate can be removed and placed in an incubator.

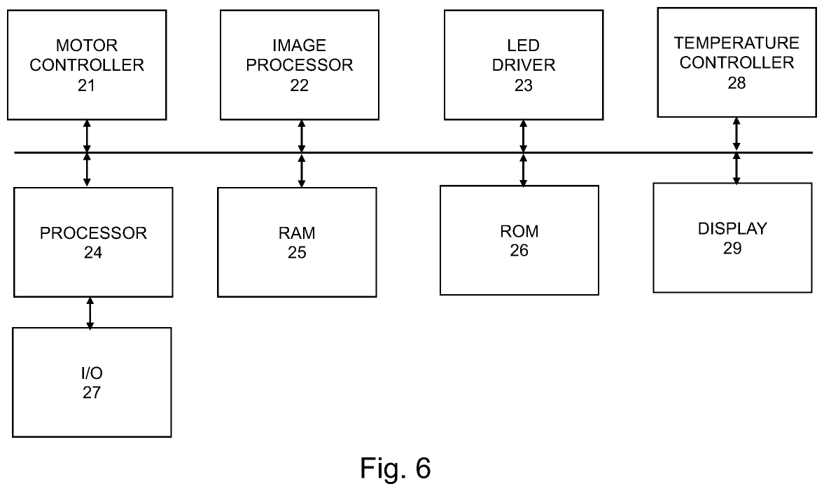

FIG. 6 is a block diagram of the circuitry for controlling the system 10. The system is run by processor 24 which is a microcontroller or microprocessor which has associated RAM 25 and ROM 26 for storage of firmware and data. The processor controls LED driver 23 which turns the LEDs on and off as required. The motor controller 21 moves the motor 15 to position the wells in an imaging position as desired by the user. In a preferred embodiment, the system can effect a quick scan of the plate in less than 1 minute and a full scan in less than 4 minutes.

The circuitry also includes a temperature controller 28 for maintaining the temperature at 98.6 degrees F. The processor 24 is connected to an I/O 27 that permits the system to be controlled by an external computer such as a laptop or desktop computer or a tablet such as an iPad or Android tablet. The connection to an external computer allows the display of the device to act as a user interface and for image processing to take place using a more powerful processor and for image storage to be done on a drive having more capacity. Alternatively, the system can include a display 29 such as a tablet mounted on one face of the system and an image processor 22 and the RAM 25 can be increased to permit the system to operate as a self-contained unit.

The image processing either on board or external, has algorithms for artificial intelligence and intelligent image analysis. The image processing permits trend analysis and forecasting, documentation and reporting, live/dead cell counts, confluence percentage and growth rates, cell distribution and morphology changes, and the percentage of differentiation.

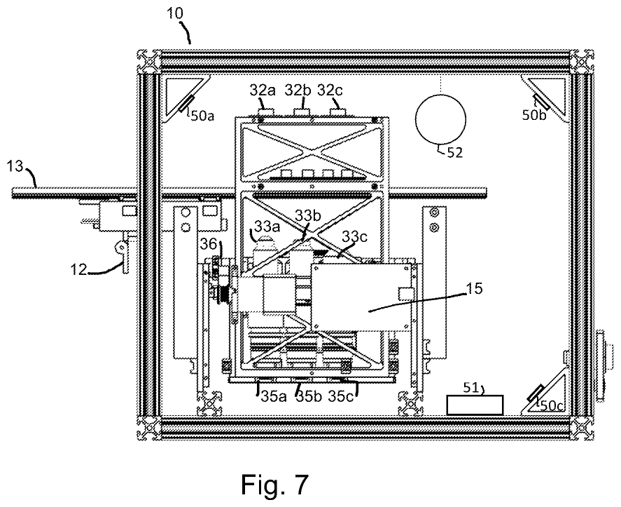

FIG. 7 shows the inside of one embodiment of the imager 10 with disinfecting equipment added to it. Three UVC LEDs 50a-50c are placed at separated locations. While three are shown it is understood that more or fewer LEDs can be used. Additionally, a rotating mirrored ball 52 is placed to reflect light from the LEDs and disperse the light throughout the inside of the imager. In some embodiments, the inner surfaces of the imager can be reflective such as by the addition of material such as mylar to aid in the light reaching the entire volume within the imager. In further embodiments, a fan or blower 51 can be placed at the lower portion of the imager to force pathogen particles to move in all directions, particularly upward, so that the particles are contacted by the UVC light. The lights, mirrors and air movers are controlled by processor 24 so that the LEDs may be activated when door 12 is closed.

In some embodiments the UVC light sources themselves can be movable within the housing, particularly for the LED UVC sources.

In other embodiments, the use of UVC light can be in conjunction with the introduction into the imager of a gas or an aerosol to generally disinfect or to disinfect for a particular pathogen. In another embodiment, the cell culture vessel that is introduced in the imager can be fitted with a battery or other power source and UVC LEDs to disinfect the local area around the culture vessel in the imager, especially when a spill has occurred.

In other embodiments, the disinfection by the use of the LEDs and mirrors, reflective surfaces, fans and/or blowers can be performed automatically when the instrument is not otherwise being used to prophylactically disinfect. The timing of the automatic disinfection can take place at a predetermined down time or can be controlled by the system processor 24. Disinfection also may be commenced by manual intervention.

In other embodiments, reflective surfaces such as mirrors that are reflecting the UVC light can be moved, e.g., tilted, rotated, moved horizontally or vertically. In other embodiments the pathogens can be moved into the path of the UVC light by use of specific frequencies of sound waves to vibrate them and/or kill them. This will be particularly effective in combination with the use of moving air caused by the use of fans and/or blowers and/or with the use of moving reflective surfaces.

In other embodiments, light frequency ranges other than UVC can be used in combination with UVC or instead of UVC to disinfect.

One or more imaging systems may be interconnected by one or more networks in any suitable form, including as a local area network (LAN) or a wide area network (WAN) such as an enterprise network or the Internet. Such networks may be based on any suitable technology and may operate according to any suitable protocol and may include wireless networks, wired networks, or fiber optic networks.

The various methods or processes outlined herein may be coded as software that is executable on one or more processors that employ any one of a variety of operating systems or platforms. Such software may be written using any of a number of suitable programming languages and/or programming or scripting tools and may be compiled as executable machine language code or intermediate code that is executed on a framework or virtual machine.

One or more algorithms for controlling methods or processes provided herein may be embodied as a readable storage medium (or multiple readable media) (e.g., a non-volatile computer memory, one or more floppy discs, compact discs (CD), optical discs, digital versatile disks (DVD), magnetic tapes, flash memories, circuit configurations in Field Programmable Gate Arrays or other semiconductor devices, or other tangible storage medium) encoded with one or more programs that, when executed on one or more computing units or other processors, perform methods that implement the various methods or processes described herein.

In various embodiments, a computer readable storage medium may retain information for a sufficient time to provide computer-executable instructions in a non-transitory form. Such a computer readable storage medium or media can be transportable, such that the program or programs stored thereon can be loaded onto one or more different computing units or other processors to implement various aspects of the methods or processes described herein. As used herein, the term “computer-readable storage medium” encompasses only a computer-readable medium that can be considered to be a manufacture (e.g., article of manufacture) or a machine. Alternatively or additionally, methods or processes described herein may be embodied as a computer readable medium other than a computer-readable storage medium, such as a propagating signal.

The terms “program” or “software” are used herein in a generic sense to refer to any type of code or set of executable instructions that can be employed to program a computing unit or other processor to implement various aspects of the methods or processes described herein. Additionally, it should be appreciated that according to one aspect of this embodiment, one or more programs that when executed perform a method or process described herein need not reside on a single computing unit or processor but may be distributed in a modular fashion amongst a number of different computing units or processors to implement various procedures or operations.

Executable instructions may be in many forms, such as program modules, executed by one or more computing units or other devices. Generally, program modules include routines, programs, objects, components, data structures, etc., that perform particular tasks or implement particular abstract data types. Typically, the functionality of the program modules may be organized as desired in various embodiments.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, e.g., to mean “including but not limited to”.

Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for disinfecting a laboratory instrument having a housing with pathogens therein, comprising the steps of: providing a plurality of UVC light sources within the housing and moving the pathogens in the housing and the light from the UVC light sources relative to each other.

2. The method of claim 1, wherein the step of moving comprises running a fan or blower to move the pathogens relative to the light from the UVC light sources.

3. The method of claim 1, wherein the step of moving comprises providing at least one mirror or one reflective surface to move the UVC light relative to the pathogens.

4. The method of claim 1, wherein the UVC light source is an LED.

5. The method of claim 1, wherein the UVC light is in the range of 200 to 280 nm.

6. The method of claim 1, wherein the UVC light is in the range of 265 to 270 nm.

7. The method of claim 1, wherein the UVC light is at 254 nm.

8. The method of claim 1, wherein the UVC light is at 222 nm.

9. An apparatus for disinfecting a laboratory instrument having a housing, comprising: a plurality of UVC light sources within the housing and at least one of a fan and a blower for moving pathogens in the housing relative to light from the UVC light sources.

10. The apparatus of claim 9, wherein the UVC light source is an LED.

11. The apparatus of claim 9, wherein the UVC light is in the range of 200 to 280 nm.

12. The apparatus of claim 9, wherein the UVC light is in the range of 265 to 270 nm.

13. The apparatus of claim 9, wherein the UVC light is at 254 nm.

14. The apparatus of claim 9, wherein the UVC light is at 222 nm.

15. An apparatus for disinfecting a laboratory instrument having a housing, comprising: a plurality of UVC light sources within the housing and at least one of a mirror and a reflective surface for moving light from the UVC light sources relative to the pathogens in the housing.

16. The apparatus of claim 15, wherein the UVC light source is an LED.

17. The apparatus of claim 15, wherein the UVC light is in the range of 200 to 280 nm.

18. The apparatus of claim 15, wherein the UVC light is in the range of 265 to 270 nm.

19. The apparatus of claim 15, wherein the UVC light is at 254 nm.

20. The apparatus of claim 15, wherein the UVC light is at 222 nm.
